(19) United States
(12) Patent Application Publication
(10) Pub. No.: US 2009/0057437 A1
(43) Pub. Date: Mar. 5, 2009

(54) Device for Dispensing Volatile Substances into the Ambient Environment

(75) Inventors: Philippe Blondeau, Paris (FR); Alice Bresson, Sannois (FR); Cedric Geffroy, Poitiers (FR)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/816,205

(22) PCT Filed: Feb. 14, 2006
(86) PCT No.: PCT/CH2006/000097

§ 371 Date: May 19, 2008

(30) Foreign Application Priority Data
Feb. 16, 2005 (EP) 05290340.8

(51) Int. Cl. A61L 9/12

(52) U.S. Cl. 239/44

(57) ABSTRACT

A device adapted to release a volatile liquid into the ambient environment. The device comprises a reservoir containing a volatile liquid in the form of two layers and a first and a second capillary element, the first capillary element being porous with respect to the liquid forming the upper layer only and the second capillary element being porous with respect to the liquid of both layers.

The present invention relates to an apparatus for dispensing volatile substances. More particularly the present invention relates to a wick-based dispensing device for the delivery of volatile substances from a bi-layer liquid to an ambient environment by evaporation.

Wick-based dispensing devices for the dispensing into the ambient environment of volatile liquids are well known in the art. One very common type of such dispensing devices consists essentially of a reservoir containing the volatile liquid and a wick immersed in the liquid and communicating with a porous substrate optionally having a broad evaporation surface. Such dispensing devices employ capillary phenomenon to provide the motive dispensing force. The wick operates to transport the liquid from the interior of the reservoir to the ambient environment, into which it evaporates. Such devices may additionally comprise auxiliary means, such as heating elements and/or fans. The liquids used in such systems consist typically of active, volatile substances, and a solvent in the case of heated devices and non-heated, fan-driven devices, or consisting of essentially water, solvent, solubilizer and active, volatile substances, when used in non-heated, non-fan-driven devices.

One problem with such systems is that, due to the formulation of the aqueous-based liquid, the wicking material tends to clog, rendering the device less effective. Moreover, no clear end of life signal (EOL) may be observed in these systems as in general a viscous residue stays in the reservoir. Furthermore, the presence of solvent, in particular in larger amounts, is costly and not environmentally friendly.

Certain attempts have recently been made to overcome these problems by using solvent and solubilizer-free aqueous-based liquid compositions, resulting in bi-layer formulations, in conventional wick-based dispensing devices. The problem with these devices is that they do not allow a concomitant evaporation of both layers.

It has now been found that it is possible to provide a device that at least substantially overcomes the problems of the art. The invention therefore provides a dispensing device adapted to release a volatile liquid into the ambient environment, the device comprising a reservoir containing a volatile liquid in the form of two layers, from which reservoir extend a first and a second capillary element, each element having a lower portion immersed in both layers of the liquid and an upper portion extending into the ambient environment, the first capillary element being porous with respect to the liquid forming the upper layer only and the second capillary element being porous with respect to the liquid of both layers and the second capillary element having on its surface a barrier layer that is impermeable to the liquid forming the upper layer.

The invention further provides a method of dispensing into an ambient environment a volatile liquid that is in the form of two layers by transporting the liquid from the reservoir to the environment by means of a first and second capillary element, each element having a lower portion immersed in both layers of the liquid and an upper portion extending into the ambient environment, the first capillary element being porous with respect to the liquid forming the upper layer only and the second capillary element being porous with respect to the liquid of both layers and the second capillary element having on its surface a barrier layer that is impermeable to the liquid forming the upper layer.

The reservoir may be made of any material that is commonly used for air-freshener devices, e.g. glass or plastic. Such materials are preferred, which are stable in the presence of fragrance materials, e.g. essential oils. From this reservoir extend the first and second capillary elements. These may be located in place by any convenient means for example, their fitting tightly within a liquid-tight plug, which blocks an orifice in the reservoir, through which the capillary elements pass. Such a plug preferably comprises a vent, which permits equalisation of pressure. Alternatively, any means for equalisation may be used, such as vented wicks as described in WO 03092750, which is incorporated herein by reference.

The barrier layer making the second capillary element impermeable to the liquid of the upper layer may take any suitable form, for example, a liquid coating applied thereto and allowed harden or dry, or a sleeve in the form of an adhesive tape or a tight-fitting tubular member of any suitable material placed thereon. The tubular member may be made, for example, of polyethylene, polypropylene, PETG (polyethylene glycol terephatalate), Barex® resins from BP Chemicals Ltd., polyester or polystyrene. The barrier layer may be such that it is impermeable to the liquid of both layers and is extended along the second capillary element to such an extent that only the liquid forming the lower layer is absorbed, that is, the barrier layer may extend only along that part of the second capillary element that passes through the liquid forming the upper layer and a sufficient distance into the liquid forming the lower layer, to allow for the lowering by evaporation of the boundary of the two layers.

In a preferred embodiment of the invention, the major part of the lateral surface of the lower portion of the second capillary element is covered by the barrier material and essentially only the cross-section of the second capillary element is in contact with the liquid having no barrier layer. By “major part” is meant that, at full liquid load, at least half of the capillary element in contact with the liquid of the lower layer, preferably ¾ of the capillary element, more preferably the complete lateral surface is covered by the barrier material. The proportions given refer to a filled device not yet in use to release a volatile liquid by evaporation.

In a further embodiment of the invention, the first capillary element may be arranged in such a way that it substantially covers the outer surface of the barrier layer of the second capillary element. The barrier layer may be such that it is extended along the second capillary element to such an extent that only the liquid forming the lower layer is absorbed, that is, the major part of the lateral surface of the upper and lower part of the second capillary element is covered by the barrier layer, essentially only the cross-sections having no barrier.

The volatile liquid forming two layers consists of an aqueous phase forming the lower layer and a hydrophobic phase forming the upper layer. The hydrophobic phase consists essentially of fragrance. By “fragrance” is meant a single fragrance material or a mixture of fragrance materials selected from such classes as acids, esters, alcohols, aldehydes, ketones, lactones, nitrites, ethers, acetates, hydrocarbons, sulfur- nitrogen- and oxygen-containing heterocyclic, polycyclic and macrocyclic compounds, as well essential oils of natural or synthetic origin. Such fragrance materials are described, for example, in S. Arctander, Perfume Flavors and Chemicals Vols. 1 and 2, Arctander, Montclair, N.J. USA 1969. The fragrance optionally may comprise odourless liquids such as benzyl benzoate, isopropylmyristate, and hydrocarbon derivatives, for example Isopar® from Exxon. The appropriate choice of the liquid not only depends on the amount in which it may be used but also on the nature of the fragrance as will be fully appreciated by the person skilled in the art. Preferably, the fragrance comprises not more than 25% by weight, preferably not more than 20% by weight of these liquids based on the total amount of the hydrophobic phase.

By “aqueous phase” is meant a hydrophilic liquid comprising at least 40% by weight, preferably 50 to 100% by weight of water, based on the total amount of the aqueous phase.

The ratio of the aqueous phase and the hydrophobic phase depends on the particular capillary elements chosen, and is chosen preferably in such a way that both phases are almost completely evaporated at the same time. The exact ratio can be easily determined by a person skilled in the art.

The first capillary element, being porous with respect to the liquid forming the upper layer, may be made of any material suitable for the transfer of the liquid forming the upper layer by capillary action from the lower portion to the upper portion of the capillary element. Examples of suitable materials include polyolefins, e.g. PE (polyethylene) and PP (polypropylene) or a combination thereof.

The second capillary element, being porous with respect to the liquid of both layers, i.e. the aqueous and the hydrophobic layer, may be made of any material suitable for the transfer by capillary action of the liquid of both layers. Examples of suitable materials include polyester, e.g. PET (polyethylenterephthalate), cellulose and cellulose acetate, and combinations thereof. Preferably the second capillary element is made of cellulose. The second capillary element may also be made of polyolefins, e.g. PE (polyethylene) and PP (polypropylene) or a combination thereof, which is further treated to make the material suitable to transfer by capillary action the liquid of both layers. This can be achieved, for example, by dipping the second capillary element made of pololefins into an aqueous solution comprising an anionic surfactant such as sodium ether sulfate, or a nonionic surfactant such as ethoxylated fatty alcohol, preferably at about 20 weight % based on the aqueous solution, resulting in an impregnated material, which is allowed to dry, preferably over night at room temperature, before use.

The shape of the capillary elements is not critical. They can be, for example, of square, rectangular, circular or annular section. The lengths of the capillary elements depend primarily on the height of the reservoir. They are chosen preferably in such a way that they reach the bottom of the reservoir and thus the entire contents of the reservoir can be evaporated.

In a further and preferred embodiment of the invention, the liquid forming the upper and/or the liquid forming the lower layer comprise(s) a dye, preferably of a distinctive color. Once the volatile liquid reaches the end point of evaporation, the first and second capillary elements have the same color, i.e. the color of the liquid forming the upper layer. This visual signal indicates to the consumers that the product is finished and must be replaced.

Suitable dyes for the hydrophobic phase may be selected from Puricolor® blue FBL5, Puricolor® blue ABL9, Puricolor® green U3 and Puricolor® yellow AYE23 from Ciba and suitable dyes for the aqueous phase may be selected from Covasol green W7039 from Wackherr and Vitasyn® orange RGL 90 from Clariant.

A device according to the present invention is particularly suitable for wick-based dispensing devices further comprising a heating element. In addition the wick-based dispensing device may have a fan.

The invention will now further be described with reference to the drawings, which depict a preferred embodiment of the invention and which are not to be construed as limiting in any way.

FIG. 1 is a vertical cross-section through an embodiment of the invention with a full charge of volatile liquid.

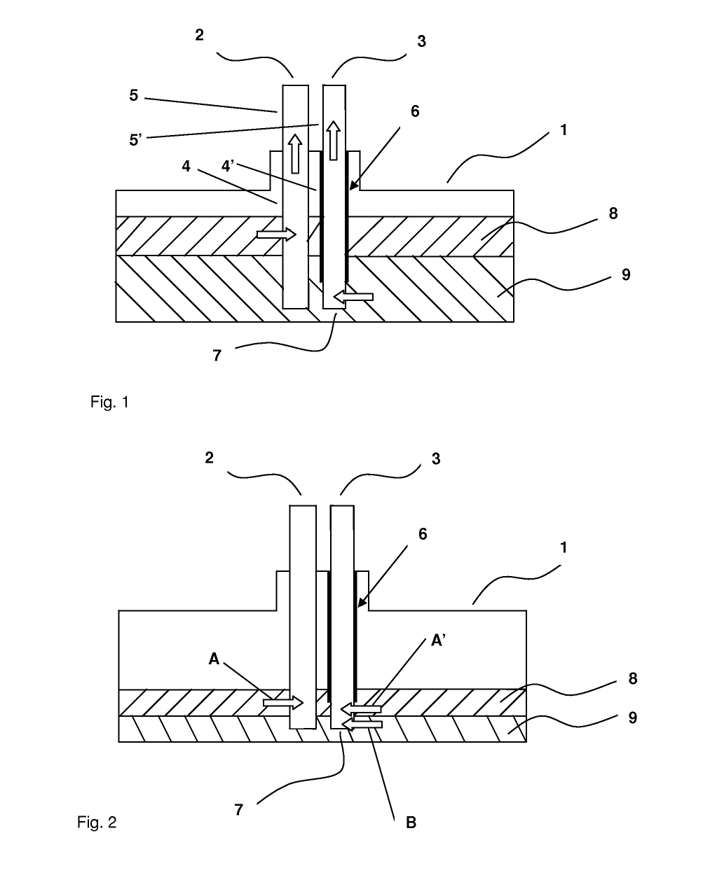

FIG. 2 is a vertical cross-section through the embodiment of FIG. 1 after evaporation of the major part of the volatile liquid.

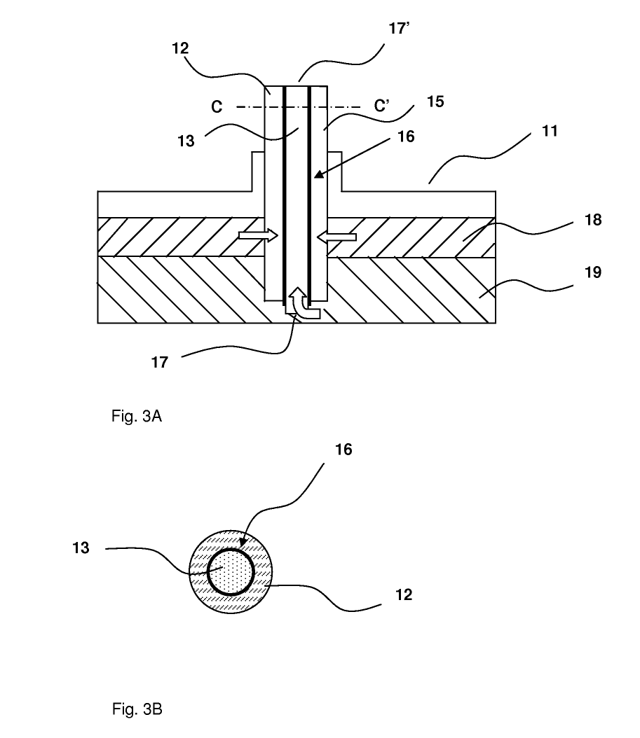

FIG. 3A is a vertical cross-section through a further embodiment of the present invention.

FIG. 3B depicts a cross-section view of the embodiment of FIG. 3A, taken along line CC′.

Referring to FIG. 1, a wick-based dispensing device comprises a reservoir (1), which contains a volatile liquid in form of an upper hydrophobic layer comprising fragrance (8) and an aqueous lower layer (9), these layers comprising different coloured dyes.

Extending from the liquid into an atmosphere through an opening in the reservoir are two capillary elements in the form of wicks (2) and (3), the wicks having upper portions, (5) and (5′) respectively, exposed to the atmosphere and lower proportions, (4) and (4′) respectively, exposed to the liquid. The materials of the wicks are selected such that the wick (2) can absorb and convey to the atmosphere only the liquid of layer (8), whereas the material of the wick (3) is capable of absorbing and conveying the liquid of both layers.

The major part of the lower portion (4′) of the wick (3) is covered by a tight-fitting polyethylene sleeve (6), making wick (3) impermeable to the liquid of the upper layer (8). At full liquid load, the sleeve (6) extends below the boundary between the layers, thus ensuring that only a lower end (7) of the lower portion (4′) of wick (3) is exposed only to liquid layer (9) and that only this liquid can evaporate through it until most of the volatile liquid is evaporated.

In operation, initially as shown in FIG. 1, upper liquid layer (8) is conveyed through and evaporates from wick (2) and lower liquid layer (9) is conveyed through the lower end (7) and evaporates from the upper portion (5′) of wick (3), as indicated by the white arrows in that drawing.

As shown in FIG. 2, this remains the case until the boundary between the layers falls below the lower end of the sleeve (6) of wick (3). At this point, both liquid layers (8) and (9) are conveyed by wick (3). This is depicted by white arrows A′ and B, whereas wick (2) continues to convey only upper liquid layer (8), as depicted by white arrow A. As a result of exposure of the lower end (7) of wick (3) to the upper liquid layer (8) and the consequent conveyance of that liquid through the wick (3), there is a noticeable change of colour of wick (3). This indicates that the device has reached the end of its life and that it should be replaced

Referring to FIG. 3A, a wick-based dispensing device is shown following the same working principle as describe in FIGS. 1 and 2. The only difference is that, instead of two individual wicks, one single wick system as also shown in further details in FIG. 3B is used. The wick-based dispensing device comprises a reservoir (11) containing a volatile liquid in form of a lower (19) and an upper liquid layer (18). Extending form the liquid into an atmosphere through an opening in the reservoir is a wick, assembled from a second capillary element (13) having on its lateral surface a tight-fitting sleeve (16) which is covered by a first capillary element (12). The material of the wick is selected such that the first capillary element (12) can absorb and convey to the atmosphere only the liquid of layer (18), whereas the material of the inner capillary element (13) is capable of absorbing and conveying both liquid layers. The outer layer (12) of the wick is a tube through which the second capillary element in form of a rode (13) made of cellulose is guided. The cellulose rod is covered by a tight-fitting sleeve (16) making it impermeable to the liquid (18) of the upper layer.

In operation, initially shown in FIG. 3A, upper liquid layer (18) is conveyed through and evaporates from the upper portion (15) of the outer layer (12) of the wick and lower liquid layer (19) is conveyed through the lower end (17) and evaporates from the upper end (17′) of the inner part (13) of the wick, as indicated by the white arrows in FIG. 3A.

The invention has been described in terms of dispensing fragrance. However, the device according to the invention may deliver other ingredients such as malodour counteracting ingredients, insect repellents, or materials having insecticidal activities, substance having antimicrobial activities, or mixtures thereof. These other ingredients may be present in both, the aqueous and/or hydrophilic phase. In addition to these active principals, fragrance compositions may comprise excipients such as dyes and UV-absorbers.

In a further embodiment of the present invention the volatile liquid forming the two layers consist of an aqueous phase comprising up to 60% by weight, preferably from 1 to 50% by weight, of ingredient(s) selected from malodour counteracting ingredients insect repellents, water soluble liquids, materials having insecticidal activities, substance having antimicrobial activities, and mixtures thereof, based on the total amount of the aqueous phase.

1-7. (canceled)

8. A device adapted to release a volatile liquid into an ambient environment, the device comprising
a reservoir containing the volatile liquid comprising a first and a second liquid layers,
a first and a second capillary element,
the first and the second capillary element each have a lower portion immersed in the reservoir and an upper portion extending into the ambient environment,
the lower portion of the first capillary element includes a porous section disposed such that only the first liquid layer is exposed to the porous section,
the lower portion of the second capillary element includes a porous section disposed such that only the second liquid layer is exposed to the porous section.

9. The device according to claim 8, wherein the second capillary element includes an impermeable sleeve for protecting the second capillary member from absorbing the first liquid layer.

10. The device according to claim 8, wherein the first and the second liquid layer each include a dye.

11. A device adapted to release a volatile liquid into an ambient environment, the device comprising
a reservoir containing the volatile liquid comprising a first and a second liquid layers,
a first and a second capillary element,
the first and the second capillary element each have a lower portion immersed ill the reservoir and an upper portion extending into the ambient environment,
the lower portion of the first capillary element includes a porous section disposed such hat only the first liquid layer is exposed to the porous section,
the lower portion of the second capillary element includes a porous section disposed such that the first liquid layer and the second liquid layer are exposed to the porous section.

12. The device according to claim 11, wherein the first and the second liquid layer each include a dye.

13. A device adapted to release a volatile liquid into an ambient environment, the device comprising
a reservoir containing the volatile liquid comprising a first and a second liquid layers,
a first capillary element surrounding a second capillary element,
the first capillary element being separated by an impermeable sleeve, the first and the second capillary element each have a lower portion immersed in the reservoir and an upper portion extending into the ambient environment,
the lower portion of the first capillary element includes a porous section disposed such that only the first liquid layer is exposed to the porous section,
the lower portion of the second capillary element is immersed in the second liquid layer and conveys liquid from the second liquid layer for evaporation through the upper portion.

14. The device according to claim 11, wherein the second capillary element is made of a material selected from polyester, cellulose and cellulose acetate, or a combination thereof.

15. The device according to claim 11, wherein the second capillary element is made of treated polyolefin, so that the material is suitable to transfer by capillary action the liquid of both layers.

16. The device according to claim 11, wherein the first capillary element is made of polyolefin.

17. The device according to claim 13, wherein the first capillary element is arranged in such a way that it substantially covers the outer surface of the second capillary element.

18. The device according to claim 13, wherein at least the upper layer of the volatile liquid comprises a dye, which once an end point of evaporation of the volatile liquid is reached results in coloration of the first and second capillary element such that both capillary elements having the same color.

19. A method of dispensing into an ambient environment a volatile liquid that is in the form of two layers comprising the steps of
providing a reservoir containing a first layer of volatile liquid and a second layer of volatile liquid,
providing a first and second capillary member extending into the reservoir at a lower portion and into the ambient environment at the upper portion,
transporting the liquid from the reservoir to the environment by the first and second capillary element,
providing the first capillary element being porous with respect to the liquid forming the upper layer, and the second capillary element being porous with respect to the liquid of both layers and the second capillary element having on its surface a barrier layer that is impermeable to the liquid forming the upper layer.
